(19) United States
(12) Patent Application Publication
(10) Pub. No.: US 2023/0410306 A1
(43) Pub. Date: Dec. 21, 2023

(54) ALIGNMENT GUIDANCE USER INTERFACE SYSTEM

(71) Applicants: Carl Zeiss Meditec, Inc., Dublin, CA (US); Carl Zeiss Meditec AG, Jena (DE)

(72) Inventor: Gregory Anderson, Dublin, CA (US)

(73) Assignees: Carl Zeiss Meditec, Inc., Dublin, CA (US); Carl Zeiss Meditec AG, Jena, TH (DE)

(21) Appl. No.: 18/035,595

(22) PCT Filed: Dec. 1, 2021
(86) PCT No.: PCT/EP2021/083764

§ 371 Date: May 5, 2023

Related U.S. Application Data
(60) Provisional application No. 63/120,525, filed on Dec. 2, 2020.

(51) Int. Cl. G06T 7/00

(52) U.S. Cl. CPC G06T 7/0014; G06T 2207/30041

(57) ABSTRACT

An ophthalmic imaging system has a specialized graphical user interface GUI to convey information for manually adjusting control inputs to bring an eye into alignment with the system. The GUI uses color and size changes to indicate axial positioning information of the system relative to a patient's eye. Furthermore, no live feed of the patient's eye is needed. Rather, a graphic indicating the patient's eye is provided and its size is controlled to indicate axial information and to filter out momentary movements of the pupil.

### FIELD OF INVENTION

The present invention is generally directed to the field of ophthalmic imaging systems. More specifically, it is directed to techniques for facilitating user operation of an ophthalmic imaging system.

### BACKGROUND

There are various type of ophthalmic examination systems, including ophthalmoscopes (or fundus cameras), Optical Coherence Tomography (OCT), and other ophthalmic imaging systems. One example of ophthalmic imaging is slit-Scanning or Broad-Line fundus imaging (see for example, U.S. Pat. Nos. 4,170,398, 4,732,466, PCT Publication No. 2012059236, US Patent Application No. 2014/0232987, and US Patent Publication No. 2015/0131050, the contents of all of which are hereby incorporated by reference), which is a technique for achieving high resolution in vivo imaging of the human retina. By illuminating a strip of the retina in a scanning fashion, the illumination stays out of the viewing path, which enables a clearer view of much more of the retina than the annular ring illumination used in traditional fundus cameras.

To obtain a good image, it is desirable for the illumination to pass unabated through the pupil and reach the fundus of an eye. This requires careful alignment of the eye with the ophthalmic imager (or other ophthalmic examination system). Various technical means have been developed to help determine the position of a patient's eye relative to the ophthalmic imaging device. However, conveying such three dimensional positioning information to a system operator (e.g., human operator or ophthalmic photographer) in an intuitive matter so that he/she may make quick use of the positioning information without requiring complex mental calculations, or mental translations from one reference plane to another, has been difficult. Generally, such systems provide a live video feed, or image/video stream, of a patient's eye on a display (viewable by the system operator) and add graphical positioning cues overlaid on the live video feed that may be interpreted by the system operator to determine positioning information of the ophthalmic imaging system relative to the patient's eye. The system operator needs to monitor the patient's eye while interpreting the system's positioning cues to determine how to adjust the position of the system and when proper alignment is achieved. Consequently, much training is generally needed to achieve a high level of competency in using such systems.

It is an object of the present invention to provide tools to facilitate the alignment of an eye with an ophthalmic examination system.

It is a further object of the present invention to provide a graphical user interface that conveys intuitive alignment information to a system operator to permit alignment of an ophthalmic imaging device to a patient's eye with reduced training.

### SUMMARY OF INVENTION

The above objects are met in a method/system for aiding a system operator to align an ophthalmic imaging device for imaging/scanning a portion of a patient's eye, such as the fundus. A preferred embodiment eliminates the need for a live feed of a patients eye. Instead various graphics, or graphic combinations, are used to convey three-dimensional (3D) information. For example, a first distinctive graphic (e.g., a dotted circle) whose size is indicative of a predefined target axial position for a pupil of an eye, is displayed/provided. A second distinctive graphic (e.g., a solid, round graphic, such as a sphere or circle) may be used to represent a patient's pupil (e.g., a pupil graphic). The displayed size of the second graphic relative to the displayed size of the first graphic is indicative of a currently observed/determined axial position of the pupil relative to the predefined target axial position (or the ophthalmic device).

Additional graphics may then be used to convey full x, y, z axis positioning information of the patient's pupil relative to the ophthalmic imaging system. In one embodiment, a cross-hair graphic may be used to convey translational (e.g., x-y axis) positioning information of the ophthalmic device relative to the pupil graphic (e.g., relative to the patient's pupil), or vice versa. z-axis information may be conveyed by illustrating the first graphic (e.g., a z-position, (round) target/reference graphic) in combination with the second graphic (e.g. the pupil graphic whose size varies with axial distance from the target graphic). For example, the size of the pupil graphic may change relative to the (e.g., fixed) size of the z-position round (target/reference) graphic, or vice versa, to represent depth information. For example, if the patient's pupil is closer to the ophthalmic device than desired (e.g., than the target z-position), the pupil graphic may be made larger than the z-position graphic, and if the patient's pupil is farther from the ophthalmic device than desired, the pupil graphic may be displayed smaller than the z-position graphic. The pupil graphic may be made to match (e.g., have a displayed size that matches) the size of the z-position graphic when the patient's pupil is within a predefined range of axial positions suitable for proper imagining. This approach of conveying depth information by use of size corresponds better to (e.g., maps much more closely to) human perception of distance/depth than other 2-dimensional guidance options.

Furthermore, since a pupil graphic is used, rather than a live feed, the size (and optionally the position) of the pupil graphic may be kept constant even while a patient's pupil momentarily moves, such as due to tremors. This eliminates unnecessary adjustments by a system operator.

Additionally, the color of the pupil graphic and/or the color of the z-position graphic may change (e.g., to match and/or to blink and/or to predefined colors or graphic patterns) to indicate when an axial position for proper alignment is achieved.

Other objects and attainments together with a fuller understanding of the invention will become apparent and appreciated by referring to the following description and claims taken in conjunction with the accompanying drawings.

Several publications may be cited or referred to herein to facilitate the understanding of the present invention. All publications cited or referred to herein, are hereby incorporated herein in their entirety by reference.

The embodiments disclosed herein are only examples, and the scope of this disclosure is not limited to them. Any embodiment feature mentioned in one claim category, e.g. system, can be claimed in another claim category, e.g. method, as well. The dependencies or references back in the attached claims are chosen for formal reasons only. However, any subject matter resulting from a deliberate reference back to any previous claims can be claimed as well, so that any combination of claims and the features thereof are disclosed and can be claimed regardless of the dependencies chosen in the attached claims.

### BRIEF DESCRIPTION OF THE DRAWINGS

Priority application U.S. Ser. No. 63/120,525 contain at least one color drawing and is herein incorporated by reference.

In the drawings wherein like reference symbols/characters refer to like parts:

FIG. 1 provide an example of a typical alignment guidance systems that provides visual cues to assist in system alignment.

FIG. 2 illustrates an exemplary enclosure for optical imaging system, such as a slit scanning ophthalmic system (as illustrated in FIG. 8).

FIG. 3 shows an example of a typical acquisition window/display/screen used for acquiring (e.g., capturing) patient images.

FIG. 4A illustrates the attachment of multiple iris cameras to an ophthalmic (or ocular) lens to facilitate alignment between an ophthalmic imaging instrument/device and a patient.

FIG. 4B shows an image collected from either the 0° or 180° iris cameras of FIG. 4A.

FIG. 4C shows an image collected from the iris camera of FIG. 4A located at 270°.

FIGS. 5A, 5B, and 5C illustrate an alternate method, in accord with the present invention, of conveying axial and translational positioning information to a system operator in an intuitive manner to achieve proper alignment of an ophthalmic imaging system to a patient's eye.

FIG. 6 illustrates a multi-step process using hard negative training for a constringed single-shot detector (SSD) in accord with the present invention.

FIG. 7 provides some exemplary pupil detection results of a model/algorithm produced by the process of FIG. 6 (e.g., ability to detect the center of the pupil), and a confidence metric between 0 and 1.

FIG. 8 illustrates an example of a slit scanning ophthalmic system for imaging a fundus.

FIG. 9 illustrates an example computer system (or computing device or computer).

### DESCRIPTION OF THE PREFERRED EMBODIMENTS

Ophthalmic photographers need to position the pupil very precisely relative to an ophthalmic imaging device when attempting to capture retinal images, or other ophthalmic images. A discussion of ophthalmic imaging devices, such as a fundus cameras, suitable for use with the present invention is provided below. Proper alignment relies on horizontal (x-axis), vertical (y-axis) and depth (z-axis) adjustments of the acquisition device relative to the patient. Given the difficulty in managing multiple planes of adjustment simultaneously, a user interface (e.g., a graphical user interface, GUI) that provides guidance as to the direction and magnitude of needed adjustments for proper alignment is generally provided to increase the ease-of-use of acquisition devices by ophthalmic photographers.

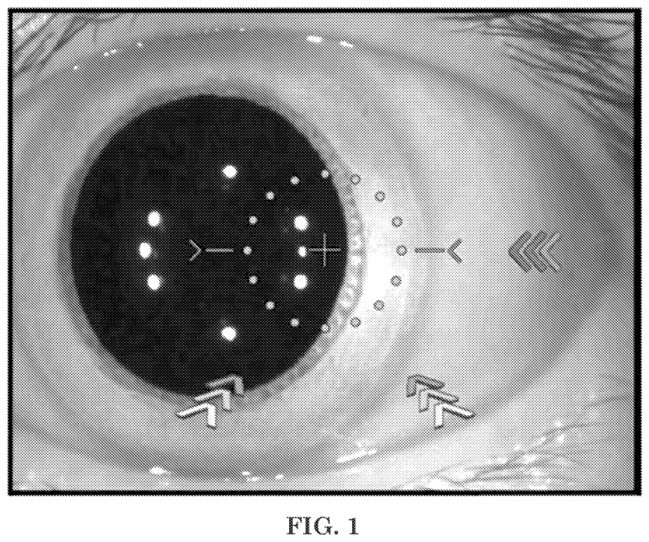

FIG. 1 provide an example of a typical alignment guidance systems that provides visual cues to assist in system alignment. The present example relies on a combination of a live view (e.g., live video feed) of a patient's eye with an overlay of arrows and color cues to convey x, y and z coordinate information of the pupil to guide a system operator to adjust system positioning to achieve correct alignment for capturing images. These systems do a poor job of communicating the z (depth) dimension to users, requiring addition cognitive steps to convert user interface (UI) elements to an understanding of the 3D relationship of the distance from the pupil to an ideal aligned position.

In contrast to the approach of FIG. 1, the present invention provides a graphic representation of a patient's eye (or target region of the eye, e.g., the pupil, iris, or symbolic eyeball), and may eliminate the use of a live feed. For example, the present invention may provide/illustrate a sphere (or solid circle) in place of a live image of an eye, and alter the size of this spherical graphic as a representation of depth, which maps much more closely to human perception of distance/depth than other 2-dimensional guidance options, such as the on-screen arrows of FIG. 1.

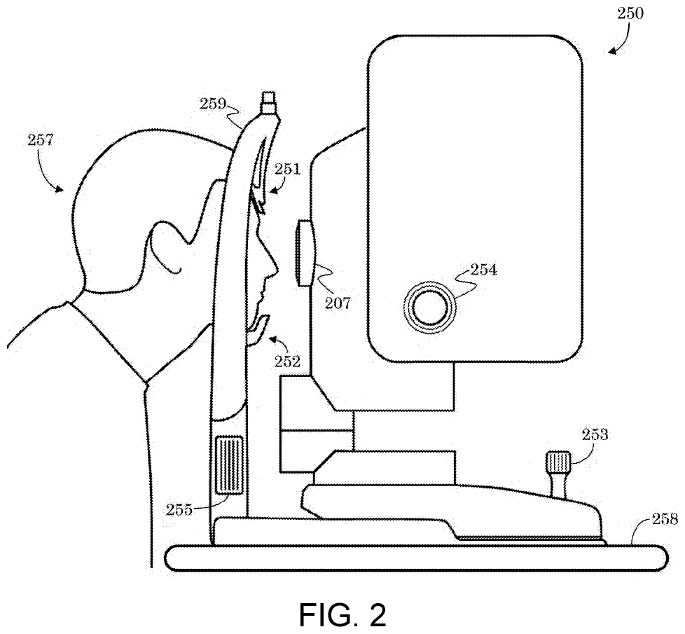

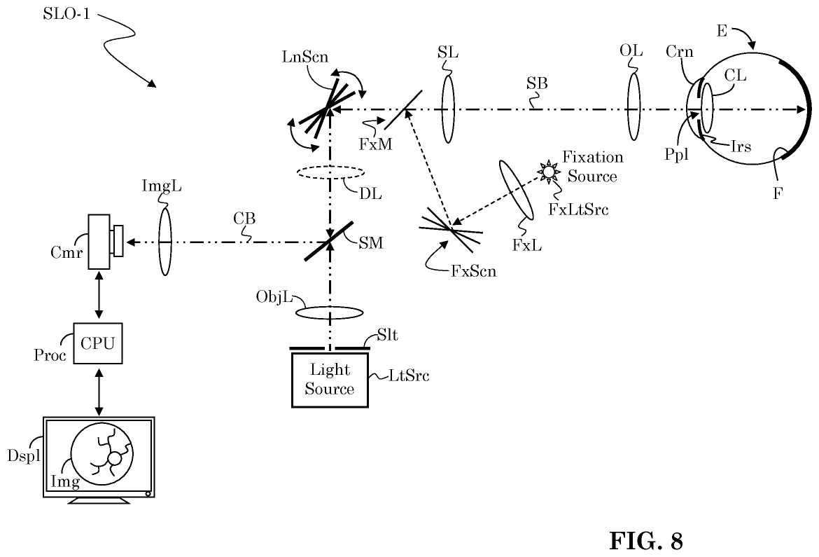

By way of example, FIG. 2 illustrates an exemplary enclosure 250 for an ophthalmic imaging system, such as a slit scanning ophthalmic system (see FIG. 8, below). Enclosure 250 (e.g., the instrument) is be positioned on a surface 258 (e.g. an adjustable table) and is coupled to a patient interface 259, which includes a headrest 251 and/or a chinrest 252 for supporting the patient (or subject) 257. Various portions of the instrument and/or patient interface can be moved relative to each other to facilitate alignment of the instrument with the subject 257 being imaged, for example, using hardware controls such as joystick 253, and knobs 254 and 255. The display (not shown in this figure) can also be mounted on the table. Ophthalmic lens 207 provides the aperture for image acquisition.

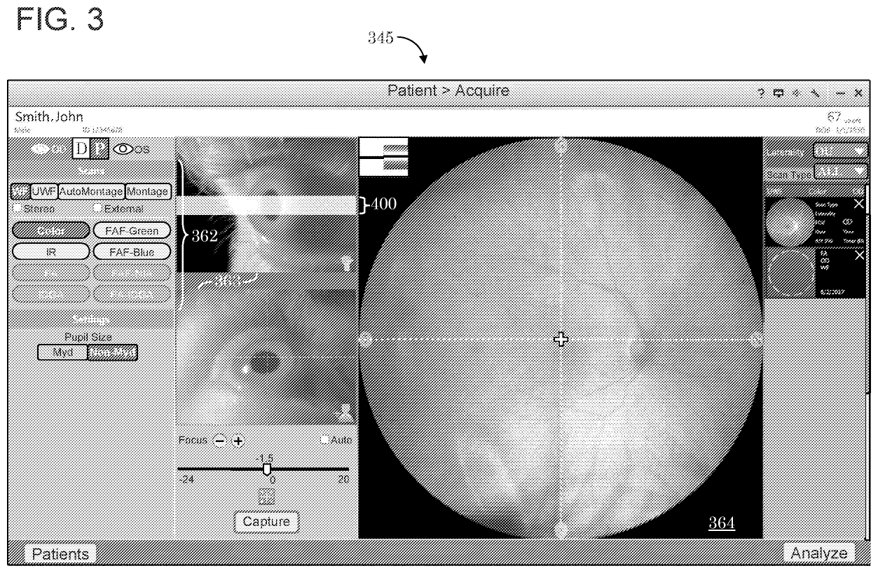

FIG. 3 shows an example of an acquisition window/display/screen 345 used for acquiring (e.g., capturing) patient images. In this window, various display elements and icons are typically displayed to the instrument operator (the user) to select the types of images to be acquired and to ensure the patient is properly aligned to the instrument. Different scan options may be displayed, such as shown in section/area 362. The scan options may include wide-field (WF), ultra-wide-field (UWF), montage of two or more images, AutoMontage, etc. Other scan options may include Color, IR (imaging using infrared light), FAF-Green (fundus auto-fluorescence with green excitation), and FAF-Blue (fundus auto-fluorescence with blue excitation). Generally, the acquisition screen 345 displays one or more pupil streams (e.g., live video streams) 363 of live images of the pupil of the eye to aid in alignment. A stream of live preview images 364 (e.g., of the fundus) may also be displayed in a section of the acquisition screen 345 to indicate the current imaging conditions. Preview images 364 may be continuously updated as alignment adjustments are made to provide the instrument user with an indication of the current image quality. Optionally, an overlay guide (semi-transparent band) 400 can be shown on the live feed image 363 to communicate an acceptable range (for example along an axial, z-axis) in which the patient's pupil can be positioned for good image acquisition.

The ophthalmic imaging device will generally include a means/mechanism for determining the position of a patient's eye relative to the ophthalmic imaging device. The specific method/mechanism for determining this relative position is not critical to the present invention, but one exemplary method is provided here.

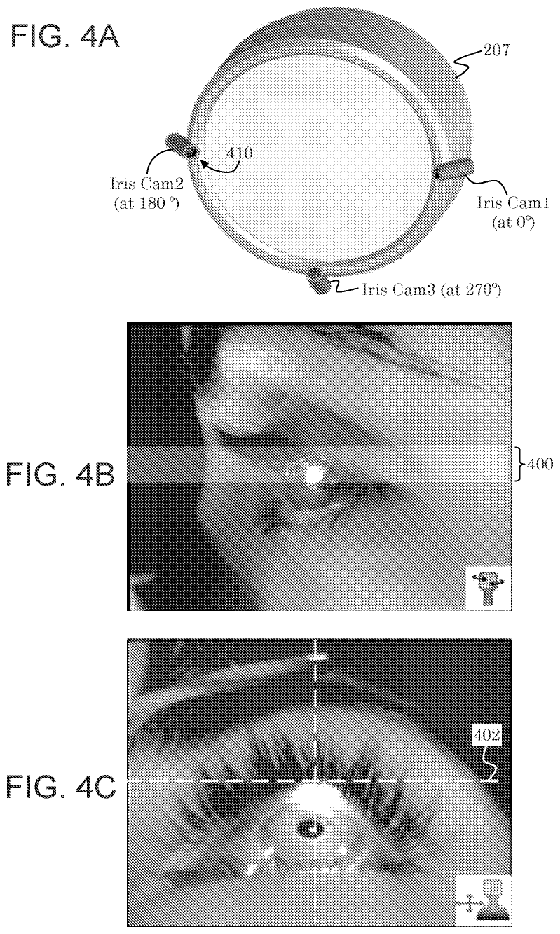

FIG. 4A illustrates the attachment of multiple iris cameras (e.g., Cam1, Cam2, and Cam3) to an ophthalmic (or ocular) lens 207 of an ophthalmic imaging system. The iris cameras are positioned to image the iris (e.g., positioned to image the exterior of the eye). This permits the collecting of images of the iris and pupil of the subject's eye, which may be used to facilitate alignment between an ophthalmic imaging instrument/device/system and a patient. In the embodiment illustrated in FIG. 4A, the iris cameras are installed roughly at 0, 180, and 270 degree positions around ophthalmic lens 207, from the patient's perspective, with respect to the ophthalmic lens 207. It is desirable for the iris cameras Cam1, Cam2, and Cam3 to work off-axis so that they do not interfere with the main optical path. The iris cameras provide live images of the patient's eye on the display (e.g., on the pupil streams 363 of acquisition screen 345 in FIG. 3). FIG. 4B shows an image collected from either the 0° or 180° iris camera (e.g., Cam1 or Cam2) while FIG. 4C shows an image collected from iris camera Cam3 located at 270°. The iris camera images presented to the user can be a composite/synthetic image showing information about offset between the desired and the current location of the patient's pupil center. Operators can use this information to center the pupil and to set the correct working distance via a cross table with respect to the patient's eye. For example, FIG. 4B shows how overlay guide (semi-transparent band) 400 can be shown on the live feed image to communicate the acceptable range in which the pupil can be positioned for good image acquisition. Similarly in FIG. 4C, dashed crosshairs 402 are displayed to indicate the desired location of the pupil for optimum imaging.

At least two iris cameras are needed to cover all three degrees of freedom (x,y,z) at any given time. Offset information is extracted by detecting the patient's pupil and locating the center of the pupil and then comparing it to stored and calibrated reference values of pupil centers. For example, iris camera Cam3 (located at the 270° position) maps the x coordinate of the patient's pupil center to the column coordinate of the iris camera image (which is comprised of rows and columns of pixels), while the z-coordinate is mapped to the row coordinate of the camera image. As the patient or the instrument moves laterally (e.g., right to left or left to right), the image moves laterally (e.g., right to left), and as the instrument is moved closer or farther away from the patient, the image of the pupil will move up or down in FIG. 4B such that the axial information is translated to, and displayed as, a vertical displacement. The y-coordinate of the patient's pupil is extracted by one or both of the iris cameras located at 0° and 180° (Cam1 and/or Cam2) as they map the y-coordinate to different rows of the image.

Although translational movement (e.g., along the x-axis and y axis) of the eye relative to cross-hairs 402 is intuitive to an operator, the use of vertical movement of the image in combination with a horizontal overlay guide 400 might not be optimal for conveying an intuitive understanding of axial motion (e.g., along the z-axis) and axial positioning information.

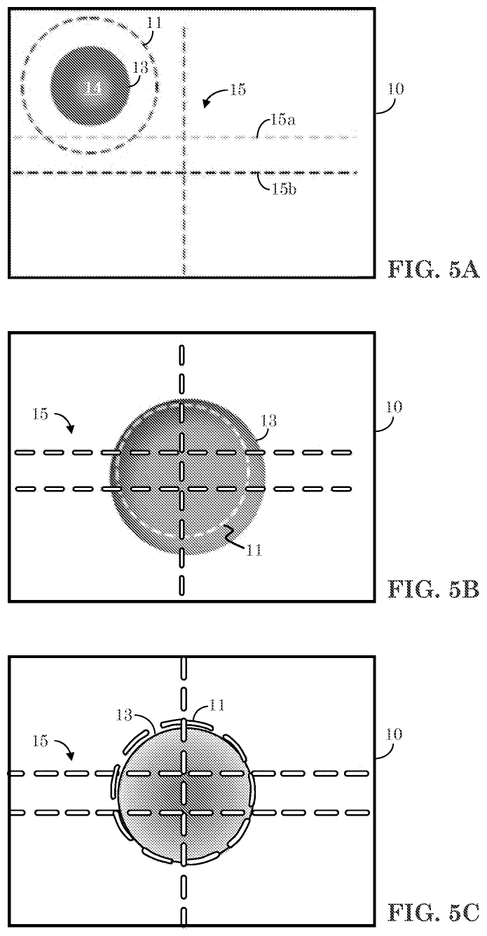

FIGS. 5A to 5C illustrate an alternate method, in accord with the present invention, of conveying axial motion and positioning information to a system operator in an intuitive manner. Rather than showing a live stream view of a patient's eye, FIG. 5A shows a graphical user interface (within a window/display/screen 10) responsive to the determined position of the ophthalmic imaging system relative to the patient's eye (or the patient). The present GUI provides a first graphic, e.g., a dotted circle, 11 whose size indicates a predefined target axial position (or target axial range) for the pupil to achieve proper imaging. Thus, the size of dotted circle 11 may be fixed if the target axial position (or target axial range) is fixed. Also provided is a second graphic, e.g., sphere 13 (or other spherical or circular shape) that represents the pupil, and whose size is dependent upon (e.g., is indicative of) a current axial position of the pupil (or patient) relative to the ophthalmic device. For example, the size of sphere 13 may be enlarged as the ophthalmic device is moved toward (e.g., closer) to the patient, or reduced as the ophthalmic device is moved away (e.g., farther) from the patient.

Optionally in an alternate embodiment, the portion of the sphere 13 that is within the predefined target axial position 11 (e.g., within the plane of dotted circle 11), may be displayed brighter (as indicated by bright spot 14) than the portion of the sphere 13 that is not within the predefined target axial position, as indicated by less bright region of sphere 13. For example, the portion of sphere 13 that is darker than bright spot 14 may be farther away from the axial position defined by dotted circle 11 and the ophthalmic device. Further alternatively, the color distribution of sphere 13 may be such that the portion of sphere 13 that is closer to the ophthalmic device is made lighter or brighter or of a different color than that the portion of sphere 13 that is farther from the ophthalmic device. In general, different colors could be used instead of different brightness levels.

A third graphic, e.g. cross-hairs 15 (or Cartesian plane, or other graphic indicative of a plane normal to the axial direction/axis), provides/indicates a predefined reference position on the plane. In the present example, the center region of the cross-hairs may indicate the predefined reference position on the plane, and a translational position of the sphere 13 on the display 10 may be indicative of a current translational position of the pupil on the plane. In the present example, cross-hairs 15 has two horizontal lines 15a and whose separation may indicate a desired positioning range for optimal imaging on the xy plan. Alternatively, double horizontal dash lines 15a/15b may be an alternate axial information indicator, as explained above in reference to the semi-transparent band 400 of FIG. 4B. Further alternatively, cross-hairs 15 may have a single horizontal line.

In the present embodiment, the center of the sphere 13 is always within the dotted circle 11, and preferably maintained aligned with the center of the dotted circle 13. In this manner, both dotted circle 11 and sphere 13 are move in tandem about the display/screen in response to translational motion (e.g. changes in the x and y axes) of the pupil, while the size, intensity, and/or color change of sphere 13 indicates its axial position relative to the dotted circle 11.

Thus, based on the positioning information provided by the GUI in FIG. 5A, the ophthalmic device would be understood to be below the pupil (e.g., along the y-axis), to the left of the pupil (along the x-axis, e.g., from the patient's perspective), and far away from the pupil along the z-axis, as is indicated by the sphere (or circle) 13 being smaller than dotted circle 11, i.e., the size guide.

With reference to FIG. 5B, the present example shows a repositioning of the ophthalmic device such that the device is now aligned in the x-y axes, as indicated by cross-hairs 15 changing to a different color than that of FIG. 5A. That is, the displayed color and/or pattern of cross-hairs 15 may change from a first color and/or pattern and/or size (e.g., small, thin, black dash lines) when the eye is not at the target translational position (as illustrated in FIG. 5A) to a second color and/or pattern and/or size (e.g., larger, thicker, green dash lines) when the ophthalmic device is aligned along the x and y axes (as illustrated in FIG. 5B). In FIG. 5B, however, sphere/circle 13 remains the same color as in FIG. 5A (e.g., red or reddish) to indicate that alignment in the z-axis has not been achieved. In the present example, sphere (or solid circle) 13, which represents the pupil, is larger than dotted circle 11 (the z-axis guide), indicating to the user that the device is too close to the pupil along the z-axis.

FIG. 5C illustrates a state where the ophthalmic device is perfectly (or satisfactorily) aligned along all three axes, as indicated cross-hairs 15, dotted circle 11, and sphere 13 all changing to be the same color (e.g., green). As illustrated, sphere 13 may not need to be exactly equal in size to circle 11 to achieve proper alignment within the z-axis. The ophthalmic system may identify a preferred (suitable) axial range for imaging, and indicate proper alignment (e.g., green color) as long as the pupil is within this identified axial range (e.g., the size of sphere 13 is within a predefined size range of dotted circle 11).

The progress in the z-axis indicator (pupil sphere 13) across FIGS. 5A to 5C (e.g., the change in size of the sphere 13 from FIG. 5A, to FIG. 5B, to FIG. 5C) shows a key difference between the present alignment guide system (e.g., GUI or method) and the typical systems discussed above. For example, no “perspective” arrows (as illustrated in FIG. 1) are required to cue the system user that the system is too close or too far away from the eye. This axial information is simply communicated via the size of the pupil representation (red or green sphere (or circle) 13) relative to the size guide (dotted circle 11). The present mechanism requires no cognitive manipulations to map arrows (or a horizontal, semi-transparent band overlay guide) or other indicators to the z-axis and does not require a live view of the eye to guide the user towards optimal alignment.

While a live view of the eye may communicate distance through observed size changes, the scale of visual, video size is too small to provide useful feedback to the user. Also, small eye movements (e.g., tremors) can be exaggerated with a close-up live view, leading to unstable feedback to the system user. The illustrated sphere 13, by contrast, is completely configurable by the system to provide scaled size and stable positioning feedback that is readily perceived and responded to by the user. Thus, since the system determines the size of the displayed sphere 13, it can filter out (or mask) momentary movements, or tremors, from the patient. For example, a timer may be provided such that if a change in distance of the pupil relative to the ophthalmic imaging device is of a duration lower than a predefined threshold, the displayed size of sphere 13 remains unchanged.

The present system may further be coupled with, and augment, an automatic imaging device alignment system. For example, the present system may provide fine tuning to the automatic image device alignment system. Or if the automatic image device alignment system requires that the device be within a specific position range of the patient's eye for proper operation, the present system may quickly bring the imaging device to within this target position range needed by the automatic image device alignment system for proper operation.

An exemplary automatic image device alignment system is herein presented.

Pupil detection is integral to alignment guidance during fundus image acquisition and automated fundus image capture. A deep learning algorithm for real-time tracking of pupils at greater than 25 frames per second (fps) is herein presented. In the present example, 13,674 eye images that provide off-axis views of patients' pupils were collected using prototype software on a CLARUS™ 500 (ZEISS, Dublin, CA). This dataset was divided into 3 parts:
Dataset1) Annotated training set containing 4,890 images with manual boundaries marked by at least one of five graders;
Dataset2) Unannotated training set with 7,000 images; and
Dataset3) Hold-out annotated test set with 784 images from 32 mydriatic and 29 non-mydriatic subjects.
   Accuracy of the algorithm was measured assuming a successful result meant localization within 400 μm of the manual annotations.

To reduce operation time, a constringed single-shot detector (SSD) inspired by single-shot multi-box detection technique (as is known in the art) was used, comprised of three feature extraction and three candidate box prediction layers. The confidence score was used to predict at most one box out of 944 candidate output boxes.

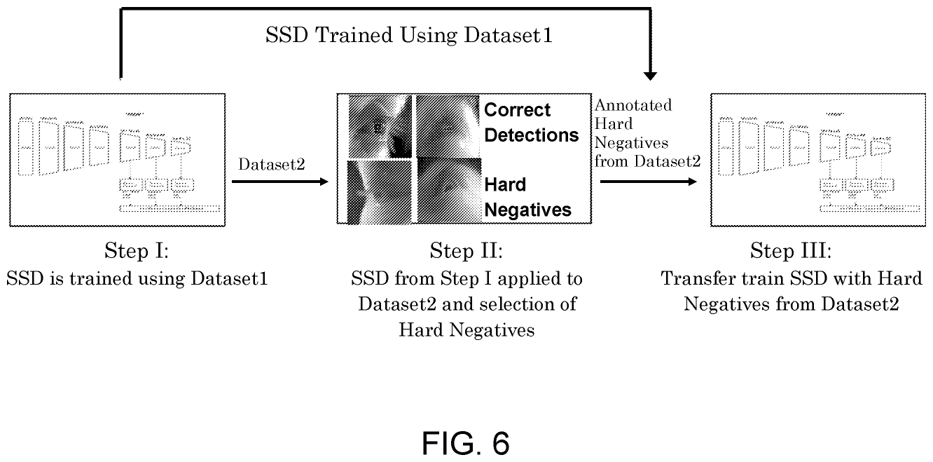

FIG. 6 illustrates the multi-step process using hard negative training for the SSD. More specifically, in step I the SSD is trained using annotated Dataset1. In step II, the trained algorithm is applied to unannotated Dataset2. From the results, severely misidentified images are manually chosen as hard negatives and annotated (692 images). In step III, the SSD trained in step I is transfer-trained using the annotated hard negatives from Dataset2.

The final model/algorithm achieved accuracies of 95.1% and 98.3% on mydriatic and non-mydriatic images of Dataset3. By comparison, the model/algorithm developed in step I (without hard-negative training) achieved accuracies of 91.7% and 95.6%.

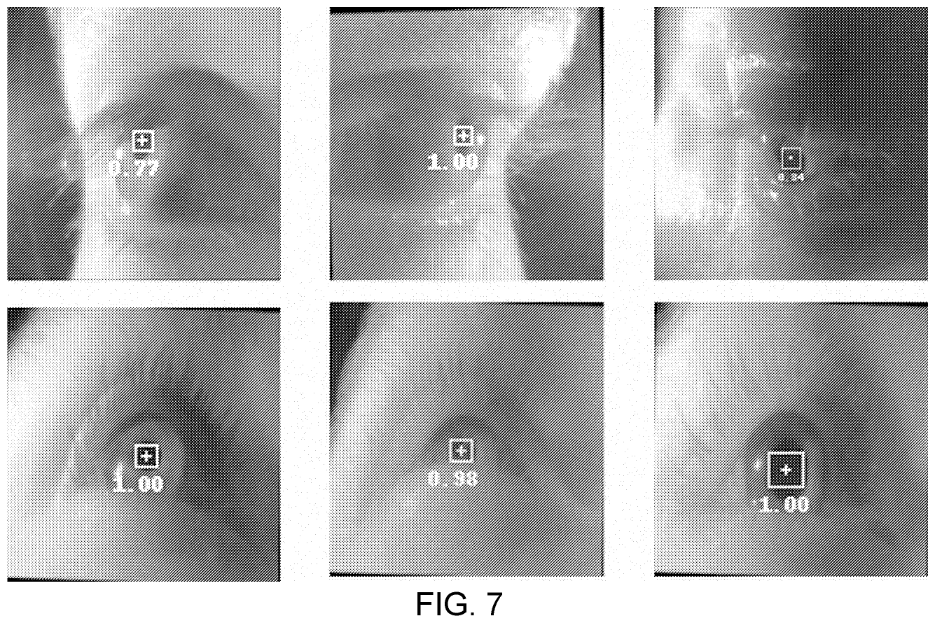

FIG. 7 provides some sample results of the present model's pupil detection results (e.g., ability to detect the center of the pupil) and a confidence metric between 0 and 1. Average execution time of the model/algorithm was 7.57 ms (132 fps) running on a Macbook™ Pro i5-7360U CPU, 34.4 ms (29 fps) running on an Intel® Core i7-6920HQ CPU, and 36.2 ms (27 fps) running on an NVIDIA nano with ARM A57.

Thus, the present model/algorithm was shown to provide robust, real-time pupil detection for alignment guidance, with accuracies greater than 95% in detecting the correct pupil location within 400 μm of manual annotations while also operating at a frame rate greater than the camera acquisition. The present GUI system may then be used to verify the present model's results and achieve greater levels of alignment.

Hereinafter is provided a description of various hardware and architectures suitable for the present invention.

Fundus Imaging System

Two categories of imaging systems used to image the fundus are flood illumination imaging systems (or flood illumination imagers) and scan illumination imaging systems (or scan imagers). Flood illumination imagers flood with light an entire field of view (FOV) of interest of a specimen at the same time, such as by use of a flash lamp, and capture a full-frame image of the specimen (e.g., the fundus) with a full-frame camera (e.g., a camera having a two-dimensional (2D) photo sensor array of sufficient size to capture the desired FOV, as a whole). For example, a flood illumination fundus imager would flood the fundus of an eye with light, and capture a full-frame image of the fundus in a single image capture sequence of the camera. A scan imager provides a scan beam that is scanned across a subject, e.g., an eye, and the scan beam is imaged at different scan positions as it is scanned across the subject creating a series of image-segments that may be reconstructed, e.g., montaged, to create a composite image of the desired FOV. The scan beam could be a point, a line, or a two-dimensional area such a slit or broad line. Examples of fundus imagers are provided in U.S. Pat. Nos. 8,967,806 and 8,998,411.

FIG. 8 illustrates an example of a slit scanning ophthalmic system SLO-1 for imaging a fundus F, which is the interior surface of an eye E opposite the eye lens (or crystalline lens) CL and may include the retina, optic disc, macula, fovea, and posterior pole. In the present example, the imaging system is in a so-called “scan-descan” configuration, wherein a scanning line beam SB traverses the optical components of the eye E (including the cornea Crn, iris Irs, pupil Ppl, and crystalline lens CL) to be scanned across the fundus F. In the case of a flood fundus imager, no scanner is needed, and the light is applied across the entire, desired field of view (FOV) at once. Other scanning configurations are known in the art, and the specific scanning configuration is not critical to the present invention. As depicted, the imaging system includes one or more light sources LtSrc, preferably a multi-color LED system or a laser system in which the etendue has been suitably adjusted. An optional slit Slt (adjustable or static) is positioned in front of the light source LtSrc and may be used to adjust the width of the scanning line beam SB. Additionally, slit Slt may remain static during imaging or may be adjusted to different widths to allow for different confocality levels and different applications either for a particular scan or during the scan for use in suppressing reflexes. An optional objective lens ObjL may be placed in front of the slit Slt. The objective lens ObjL can be any one of state-of-the-art lenses including but not limited to refractive, diffractive, reflective, or hybrid lenses/systems. The light from slit Slt passes through a pupil splitting mirror SM and is directed towards a scanner LnScn. It is desirable to bring the scanning plane and the pupil plane as near together as possible to reduce vignetting in the system. Optional optics DL may be included to manipulate the optical distance between the images of the two components. Pupil splitting mirror SM may pass an illumination beam from light source LtSrc to scanner LnScn, and reflect a detection beam from scanner LnScn (e.g., reflected light returning from eye E) toward a camera Cmr. A task of the pupil splitting mirror SM is to split the illumination and detection beams and to aid in the suppression of system reflexes. The scanner LnScn could be a rotating galvo scanner or other types of scanners (e.g., piezo or voice coil, micro-electromechanical system (MEMS) scanners, electro-optical deflectors, and/or rotating polygon scanners). Depending on whether the pupil splitting is done before or after the scanner LnScn, the scanning could be broken into two steps wherein one scanner is in an illumination path and a separate scanner is in a detection path. Specific pupil splitting arrangements are described in detail in U.S. Pat. No. 9,456,746, which is herein incorporated in its entirety by reference.

From the scanner LnScn, the illumination beam passes through one or more optics, in this case a scanning lens SL and an ophthalmic or ocular lens OL, that allow for the pupil of the eye E to be imaged to an image pupil of the system. Generally, the scan lens SL receives a scanning illumination beam from the scanner LnScn at any of multiple scan angles (incident angles), and produces scanning line beam SB with a substantially flat surface focal plane (e.g., a collimated light path). Ophthalmic lens OL may then focus the scanning line beam SB onto an object to be imaged. In the present example, ophthalmic lens OL focuses the scanning line beam SB onto the fundus F (or retina) of eye E to image the fundus. In this manner, scanning line beam SB creates a traversing scan line that travels across the fundus F. One possible configuration for these optics is a Kepler type telescope wherein the distance between the two lenses is selected to create an approximately telecentric intermediate fundus image (4-f configuration). The ophthalmic lens OL could be a single lens, an achromatic lens, or an arrangement of different lenses. All lenses could be refractive, diffractive, reflective or hybrid as known to one skilled in the art. The focal length(s) of the ophthalmic lens OL, scan lens SL and the size and/or form of the pupil splitting mirror SM and scanner LnScn could be different depending on the desired field of view (FOV), and so an arrangement in which multiple components can be switched in and out of the beam path, for example by using a flip in optic, a motorized wheel, or a detachable optical element, depending on the field of view can be envisioned. Since the field of view change results in a different beam size on the pupil, the pupil splitting can also be changed in conjunction with the change to the FOV. For example, a 45° to 60° field of view is a typical, or standard, FOV for fundus cameras. Higher fields of view, e.g., a widefield FOV, of 60°-120°, or more, may also be feasible. A widefield FOV may be desired for a combination of the Broad-Line Fundus Imager (BLFI) with another imaging modalities such as optical coherence tomography (OCT). The upper limit for the field of view may be determined by the accessible working distance in combination with the physiological conditions around the human eye. Because a typical human retina has a FOV of 140° horizontal and 80°-100° vertical, it may be desirable to have an asymmetrical field of view for the highest possible FOV on the system.

The scanning line beam SB passes through the pupil Ppl of the eye E and is directed towards the retinal, or fundus, surface F. The scanner LnScn1 adjusts the location of the light on the retina, or fundus, F such that a range of transverse locations on the eye E are illuminated. Reflected or scattered light (or emitted light in the case of fluorescence imaging) is directed back along as similar path as the illumination to define a collection beam CB on a detection path to camera Cmr.

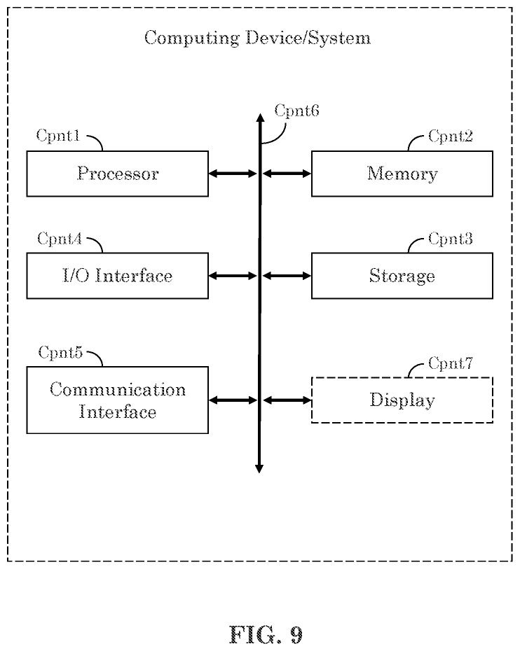

In the “scan-descan” configuration of the present, exemplary slit scanning ophthalmic system SLO-1, light returning from the eye E is “descanned” by scanner LnScn on its way to pupil splitting mirror SM. That is, scanner LnScn scans the illumination beam from pupil splitting mirror SM to define the scanning illumination beam SB across eye E, but since scanner LnScn also receives returning light from eye E at the same scan position, scanner LnScn has the effect of descanning the returning light (e.g., cancelling the scanning action) to define a non-scanning (e.g., steady or stationary) collection beam from scanner LnScn to pupil splitting mirror SM, which folds the collection beam toward camera Cmr. At the pupil splitting mirror SM, the reflected light (or emitted light in the case of fluorescence imaging) is separated from the illumination light onto the detection path directed towards camera Cmr, which may be a digital camera having a photo sensor to capture an image. An imaging (e.g., objective) lens ImgT may be positioned in the detection path to image the fundus to the camera Cmr. As is the case for objective lens ObjL, imaging lens ImgL may be any type of lens known in the art (e.g., refractive, diffractive, reflective or hybrid lens). Additional operational details, in particular, ways to reduce artifacts in images, are described in PCT Publication No. WO2016/124644, the contents of which are herein incorporated in their entirety by reference. The camera Cmr captures the received image, e.g., it creates an image file, which can be further processed by one or more (electronic) processors or computing devices (e.g., the computer system of FIG. 9). Thus, the collection beam (returning from all scan positions of the scanning line beam SB) is collected by the camera Cmr, and a full-frame image Img may be constructed from a composite of the individually captured collection beams, such as by montaging. However, other scanning configuration are also contemplated, including ones where the illumination beam is scanned across the eye E and the collection beam is scanned across a photo sensor array of the camera. PCT Publication WO 2012/059236 and US Patent Publication No. 2015/0131050, herein incorporated by reference, describe several embodiments of slit scanning ophthalmoscopes including various designs where the returning light is swept across the camera's photo sensor array and where the returning light is not swept across the camera's photo sensor array.

In the present example, the camera Cmr is connected to a processor (e.g., processing module) Proc and a display (e.g., displaying module, computer screen, electronic screen, etc.) Dspl, both of which can be part of the image system itself, or may be part of separate, dedicated processing and/or displaying unit(s), such as a computer system wherein data is passed from the camera Cmr to the computer system over a cable or computer network including wireless networks. The display and processor can be an all in one unit. The display can be a traditional electronic display/screen or of the touch screen type and can include a user interface for displaying information to and receiving information from an instrument operator, or user. The user can interact with the display using any type of user input device as known in the art including, but not limited to, mouse, knobs, buttons, pointer, and touch screen.

It may be desirable for a patient's gaze to remain fixed while imaging is carried out. One way to achieve this is to provide a fixation target that the patient can be directed to stare at. Fixation targets can be internal or external to the instrument depending on what area of the eye is to be imaged. One embodiment of an internal fixation target is shown in FIG. 8. In addition to the primary light source LtSrc used for imaging, a second optional light source FxLtSrc, such as one or more LEDs, can be positioned such that a light pattern is imaged to the retina using lens FxL, scanning element FxScn and reflector/mirror FxM. Fixation scanner FxScn can move the position of the light pattern and reflector FxM directs the light pattern from fixation scanner FxScn to the fundus F of eye E. Preferably, fixation scanner FxScn is position such that it is located at the pupil plane of the system so that the light pattern on the retina/fundus can be moved depending on the desired fixation location.

Slit-scanning ophthalmoscope systems are capable of operating in different imaging modes depending on the light source and wavelength selective filtering elements employed. True color reflectance imaging (imaging similar to that observed by the clinician when examining the eye using a hand-held or slit lamp ophthalmoscope) can be achieved when imaging the eye with a sequence of colored LEDs (red, blue, and green). Images of each color can be built up in steps with each LED turned on at each scanning position or each color image can be taken in its entirety separately. The three, color images can be combined to display the true color image, or they can be displayed individually to highlight different features of the retina. The red channel best highlights the choroid, the green channel highlights the retina, and the blue channel highlights the anterior retinal layers. Additionally, light at specific frequencies (e.g., individual colored LEDs or lasers) can be used to excite different fluorophores in the eye (e.g., autofluorescence) and the resulting fluorescence can be detected by filtering out the excitation wavelength.

The fundus imaging system can also provide an infrared reflectance image, such as by using an infrared laser (or other infrared light source). The infrared (IR) mode is advantageous in that the eye is not sensitive to the IR wavelengths. This may permit a user to continuously take images without disturbing the eye (e.g., in a preview/alignment mode) to aid the user during alignment of the instrument. Also, the IR wavelengths have increased penetration through tissue and may provide improved visualization of choroidal structures. In addition, fluorescein angiography (FA) and indocyanine green (ICG) angiography imaging can be accomplished by collecting images after a fluorescent dye has been injected into the subject's bloodstream. For example, in FA (and/or ICG) a series of time-lapse images may be captured after injecting a light-reactive dye (e.g., fluorescent dye) into a subject's bloodstream. It is noted that care must be taken since the fluorescent dye may lead to a life-threatening allergic reaction in a portion of the population. High contrast, greyscale images are captured using specific light frequencies selected to excite the dye. As the dye flows through the eye, various portions of the eye are made to glow brightly (e.g., fluoresce), making it possible to discern the progress of the dye, and hence the blood flow, through the eye.

Computing Device/System

FIG. 9 illustrates an example computer system (or computing device or computer device). In some embodiments, one or more computer systems may provide the functionality described or illustrated herein and/or perform one or more steps of one or more methods described or illustrated herein. The computer system may take any suitable physical form. For example, the computer system may be an embedded computer system, a system-on-chip (SOC), a single-board computer system (SBC) (such as, for example, a computer-on-module (COM) or system-on-module (SOM)), a desktop computer system, a laptop or notebook computer system, a mesh of computer systems, a mobile telephone, a personal digital assistant (PDA), a server, a tablet computer system, an augmented/virtual reality device, or a combination of two or more of these. Where appropriate, the computer system may reside in a cloud, which may include one or more cloud components in one or more networks.

In some embodiments, the computer system may include a processor Cpnt1, memory Cpnt2, storage Cpnt3, an input/output (I/O) interface Cpnt4, a communication interface Cpnt5, and a bus Cpnt6. The computer system may optionally also include a display Cpnt7, such as a computer monitor or screen.

Processor Cpnt1 includes hardware for executing instructions, such as those making up a computer program. For example, processor Cpnt1 may be a central processing unit (CPU) or a general-purpose computing on graphics processing unit (GPGPU). Processor Cpnt1 may retrieve (or fetch) the instructions from an internal register, an internal cache, memory Cpnt2, or storage Cpnt3, decode and execute the instructions, and write one or more results to an internal register, an internal cache, memory Cpnt2, or storage Cpnt3. In particular embodiments, processor Cpnt1 may include one or more internal caches for data, instructions, or addresses. Processor Cpnt1 may include one or more instruction caches, one or more data caches, such as to hold data tables. Instructions in the instruction caches may be copies of instructions in memory Cpnt2 or storage Cpnt3, and the instruction caches may speed up retrieval of those instructions by processor Cpnt1. Processor Cpnt1 may include any suitable number of internal registers, and may include one or more arithmetic logic units (ALUs). Processor Cpnt1 may be a multi-core processor; or include one or more processors Cpnt1. Although this disclosure describes and illustrates a particular processor, this disclosure contemplates any suitable processor.

Memory Cpnt2 may include main memory for storing instructions for processor Cpnt1 to execute or to hold interim data during processing. For example, the computer system may load instructions or data (e.g., data tables) from storage Cpnt3 or from another source (such as another computer system) to memory Cpnt2. Processor Cpnt1 may load the instructions and data from memory Cpnt2 to one or more internal register or internal cache. To execute the instructions, processor Cpnt1 may retrieve and decode the instructions from the internal register or internal cache. During or after execution of the instructions, processor Cpnt1 may write one or more results (which may be intermediate or final results) to the internal register, internal cache, memory Cpnt2 or storage Cpnt3. Bus Cpnt6 may include one or more memory buses (which may each include an address bus and a data bus) and may couple processor Cpnt1 to memory Cpnt2 and/or storage Cpnt3. Optionally, one or more memory management unit (MMU) facilitate data transfers between processor Cpnt1 and memory Cpnt2. Memory Cpnt2 (which may be fast, volatile memory) may include random access memory (RAM), such as dynamic RAM (DRAM) or static RAM (SRAM). Storage Cpnt3 may include long-term or mass storage for data or instructions. Storage Cpnt3 may be internal or external to the computer system, and include one or more of a disk drive (e.g., hard-disk drive, HDD, or solid-state drive, SSD), flash memory, ROM, EPROM, optical disc, magneto-optical disc, magnetic tape, Universal Serial Bus (USB)-accessible drive, or other type of non-volatile memory.

I/O interface Cpnt4 may be software, hardware, or a combination of both, and include one or more interfaces (e.g., serial or parallel communication ports) for communication with I/O devices, which may enable communication with a person (e.g., user). For example, I/O devices may include a keyboard, keypad, microphone, monitor, mouse, printer, scanner, speaker, still camera, stylus, tablet, touch screen, trackball, video camera, another suitable I/O device, or a combination of two or more of these.

Communication interface Cpnt5 may provide network interfaces for communication with other systems or networks. Communication interface Cpnt5 may include a Bluetooth interface or other type of packet-based communication. For example, communication interface Cpnt5 may include a network interface controller (NIC) and/or a wireless NIC or a wireless adapter for communicating with a wireless network. Communication interface Cpnt5 may provide communication with a WI-FI network, an ad hoc network, a personal area network (PAN), a wireless PAN (e.g., a Bluetooth WPAN), a local area network (LAN), a wide area network (WAN), a metropolitan area network (MAN), a cellular telephone network (such as, for example, a Global System for Mobile Communications (GSM) network), the Internet, or a combination of two or more of these.

Bus Cpnt6 may provide a communication link between the above-mentioned components of the computing system. For example, bus Cpnt6 may include an Accelerated Graphics Port (AGP) or other graphics bus, an Enhanced Industry Standard Architecture (EISA) bus, a front-side bus (FSB), a HyperTransport (HT) interconnect, an Industry Standard Architecture (ISA) bus, an InfiniBand bus, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or other suitable bus or a combination of two or more of these.

Although this disclosure describes and illustrates a particular computer system having a particular number of particular components in a particular arrangement, this disclosure contemplates any suitable computer system having any suitable number of any suitable components in any suitable arrangement.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

While the invention has been described in conjunction with several specific embodiments, it is evident to those skilled in the art that many further alternatives, modifications, and variations will be apparent in light of the foregoing description. Thus, the invention described herein is intended to embrace all such alternatives, modifications, applications and variations as may fall within the spirit and scope of the appended claims.

1. A graphical user interface (GUI) system for an ophthalmic device, comprising:
a first graphic whose size is indicative of a predefined target axial position for a pupil of an eye;
a second graphic whose size is indicative of a current axial position of the pupil;
wherein the size of the second graphic relative to the size of the first graphic is indicative of a determined axial displacement of the position of the pupil relative to the predefined target axial position.

2. The system of claim 1, wherein the size of the first graphic is fixed.

3. The system of claim 1, wherein the size of the second graphic is made equal to a target size in response to the current position of the pupil being within a predefined range of axial displacement relative to the target axial position.

4. The system of claim 3, wherein the target size is substantially equal the size of the first graphic.

5. The system of claim 1, wherein the size of the second graphic is made larger than the size of the first graphic in response to the current position of the pupil being determined to be offset from the target axial position along an axial direction toward the ophthalmic device.

6. The system of claim 5, wherein the size of the second graphic is made smaller than the size of the first graphic in response to the current position of the pupil being determined to be offset from the target axial position along an axial direction away from ophthalmic device.

7. The system of claim 1, wherein the first graphic has a first color in response to the current axial position of the pupil being determined to match the predefined target axial position, and has a second color, different than the first color, in response the current axial position of the pupil being determined to not match the predefined target axial position.

8. The system of claim 1, wherein a translational position of the second graphic on a display is indicative of a current translational position of the pupil on a plane normal to the axial direction and relative to a predefined reference position on the plane.

9. The system of claim 8, wherein the second graphic has a first color in response to the current position of the pupil in an x-y-z space being determined to match a predefined positioning range within the predefined target axial position and a predefined translational position, and has a second color, different than the first color, in response the current axial position of the pupil being determined to be within the predefined positioning range but not within a predefined translational position.

10. The system of claim 8, wherein the first graphic moves to continuously track the current position of the second graphic.

11. The system of claim 10, wherein the center of the first graphic is maintained aligned with the center of the second graphic, whereby both graphics move in tandem on the display.

12. The system of claim 1, wherein the first and second graphics are round.

13. The system of claim 12 wherein the first graphic has a transparent interior and the second graphic has an opaque interior.

14. The system of claim 13, wherein:
the second graphic is spherical;
the portion of the second graphic that is within the predefined target axial position is displayed with a first color; and
the portion of the second graphic that is not within the predefined target axial position is displayed with a second color different than the first color.

15. The system of claim 13, wherein:
the second graphic is spherical; and
the portion of the second graphic that is within the predefined target axial position is displayed brighter than the portion of the second graphic that is not within the predefined target axial position.

16. The system of claim 1, wherein the size of second graphic is adjusted to be closer to the size of the first graphic as the alignment of the device is adjusted to be closer to the predefined target axial position.
